(19) United States
(12) Patent Application Publication
(10) Pub. No.: US 2017/0319456 A1
(43) Pub. Date: Nov. 9, 2017

(54) HAIR COLOURANTS COMPRISING TETRAAMINOPYRIMIDINE DERIVATES

(71) Applicant: Henkel AG & Co. KGaA, Duesseldorf (DE)

(72) Inventors: Antje Gebert-Schwarzwaelder, Neuss (DE); Helmut Giesa, Meerbusch (DE); Annika Koenen, Grevenbroich (DE)

(73) Assignee: Henkel AG & Co. KGaA, Duesseldorf (DE)

(21) Appl. No.: 15/533,711

(22) PCT Filed: Oct. 30, 2015
(86) PCT No.: PCT/EP2015/075199

§ 371 Date: Jun. 7, 2017

(30) Foreign Application Priority Data
Dec. 10, 2014 (DE) 10 2014 225 380.2

(51) Int. Cl. A61K 8/49; A61K 8/22; A61Q 5/06; A61Q 5/08

(52) U.S. Cl. CPC A61K 8/4953; A61Q 5/08; A61K 8/22; A61Q 5/065; A61K 2800/882

(57) ABSTRACT

The present disclosure relates to compositions for colouring keratin fibres, in particular human hair, where the composition comprises, in a cosmetic carrier, at least one neutral tetraaminopyrimidine derivate of the general formula (I), (I) in which R1, R2 independently of one another are a group of the formulae (II), (III) or (IV) —(CH₂)ₙ— (II) —(CH₂)ₘ—O—(CH₂)ₚ— (III) —(CH₂)ₘ—N(R3)-(CH₂)ₚ— (IV) n is 2, 3, 4, 5 or 6, m, p in each case independently of one another are 1, 2 or 3, Y is —OH, —NH₂ or —NH-alkyl, R3 is a hydrogen atom, a C₁-C₆-alkyl group or is a C₂-C₆-alkenyl group.

### CROSS-REFERENCE TO RELATED APPLICATION

This application is a U.S. National-Stage entry under 35 U.S.C §371 based on International Application No. PCT/EP2015/075199, filed Oct. 30, 2015 which was published under PCT Article 21(2) and which claims priority to 10 2014 225 380.2, filed Dec. 10, 2014, which are all hereby incorporated in their entirety by reference

### TECHNICAL FIELD

This application pertains to a material for changing the color of keratin-containing fibers, especially human hair, the material containing certain tetraaminopyrimidine derivatives in combination with certain coupler components.

The present disclosure furthermore relates to the use of the developer-coupler combination in dyes for improving the dyeing result.

### BACKGROUND

The change in the form and color of the hair represents an important area of modern cosmetics. For the fashionable color design of hairstyles or for laminating gray or white hair with fashionable or natural hues, the consumer uses color-changing compositions.

To provide color-changing cosmetic materials for skin or keratin-containing fibers, such as human hair, someone of ordinary skill in the art knows various systems depending on the requirements of the dyeing and of the change in color.

So-called oxidation dyes are used for permanent, intensive dyeings with corresponding genuineness properties. Such dyes usually contain oxidation dye precursors, the so-called developer components and coupler components. Under the influence of oxidizing agents or of oxygen from the air, the developer components form the actual dyes among one another or by coupling with one or more of the coupling components Oxidation dyes are distinguished by intensive, outstanding, long-lasting dyeing results. For naturally acting dyeings, a mixture of a larger number of precursors of oxidation dyes can be used and, in many cases, further additional, substantive dyes are used for nuancing.

In spite of their advantageous color properties, especially oxidative hair dyes frequently are subject to disadvantages for the user, so that there is a constant need for further development of precursors of oxidation dyes.

In the search for oxidation dye precursors with a good tolerability profile, many compounds have been investigated, but frequently suffer from application-related problems, particularly from a lack of gray coverage capability. In addition to this, despite the already highly developed color systems, there still is a need for color systems, which achieve excellent luminosity and intensity of the dyeings, but at the same time, have a very good durability, very good fastness properties and an excellent homogeneity.

There is a need for improvement, especially in the field of dyeings in the natural shade range.

Many well-known color systems, with which naturally appearing color nuances can be achieved, do not have satisfactory wash fastness and do not have sufficient equalizing ability.

The publication DE 39 29 333 A1 discloses oxidation dyes containing histidine, which are toxicologically and dermatologically harmless.

### BRIEF SUMMARY

Compositions for dyeing keratinic fibers and methods of using such compositions are provided herein. In an exemplary embodiment, a composition for dyeing keratinic fibers includes, in a cosmetic carrier, at least one neutral tetraaminopyrimidine derivative of the general Formula (I):

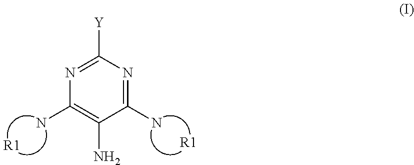

in which

R1, R2 independently of one another, represent one of the groups of formulas (II), (III) or (IV)

—(CH2)n- (II)

—(CH2)m-O—(CH2)p- (III)

—(CH2)m-N(R3)-(CH2)p (IV)
n represents 2, 3, 4, 5 or 6,
m, p independently of one another represent 1, 2 or 3,
Y represents —OH, —NH2 or —NH-alkyl,
R3 represents a hydrogen atom, a C1-C6 alkyl group or a C2-C6 alkenyl group.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It is therefore an object of the present disclosure to reduce the abovementioned disadvantages of oxidative hair dyes. The dyes are to produce intense dyeings of high colorfulness and with a good resistance to external influences, in particular, with good light fastness and fastness to washing, and which do not suffer from color fading or color shifting even after the hair is shampooed repeatedly. In addition, the selectivity of the dyeings should be as low as possible so that uniform color results are achieved on hair, which has been pretreated differently. In addition, the dyes should have a toxicologically advantageous profile.

A further object of the present disclosure is to find suitable, toxicologically and dermatologically harmless coupler components for tetraaminopyrimidine derivatives, with which a multitude of color nuances, in particular natural color nuances, can be achieved and which have outstanding fastness to washing and a particularly good equalizing ability.

Finally, it is desirable to achieve the greatest possible nuancing of the individual color shades.

It has been found that certain tetraaminopyrimidine derivatives are outstandingly suitable as oxidation dye precursors for coloring keratin-containing fibers. Particularly in conjunction with certain coupler components, colorations with good fastness properties, a high color intensity and excellent brilliance as well as excellent equalization capabilities are obtained.

A first object of the present disclosure is therefore a composition for dyeing keratinic fibers, in particular human hair, which is characterized in that it contains, in a cosmetic carrier, at least one neutral tetraaminopyrimidine derivative of the general formula (I).

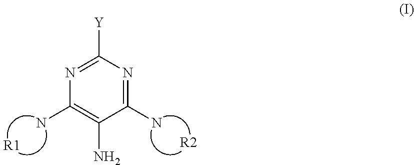

in which
R1, R2 independently of one another, represent formulas (II), (III) or (IV)

—(CH₂)ₙ— (II)

—(CH₂)ₘ—O—(CH₂)ₚ— (III)

—(CH₂)ₘ—N(R3)-(CH₂)ₚ— (IV)

n represents 2, 3, 4, 5 or 6,
m, p independently of one another represent 1, 2 or 3,
Y represents —OH, —NH₂ or —NH-alkyl,
R3 represents a hydrogen atom, a C₁-C₆ alkyl group or a C₂-C₆ alkenyl group.

Keratinic fibers are understood to be wool, fur, feathers and especially human hair. The inventive dyes can, however, also basically be used to dye other natural fibers such as, for example, cotton, jute, sisal, linen or silk, modified natural fibers, for example regenerated cellulose and nitro-, alkyl- or hydroxyalkyl- or acetylcellulose.

The inventive compositions contain the compound(s) of Formula (I) in a cosmetic carrier, preferably in an suitable aqueous, alcoholic or aqueous-alcoholic carrier. For the purpose of dyeing hair, such carriers are, for example, creams, emulsions, gels or also surfactant-containing foaming solutions, such as, for example, shampoos, foam aerosols, foam formulations or other preparations, which are suitable for application on the hair. It is, however, also conceivable to integrate the compounds of Formula (I) in a powdery or also tablet formulation.

In the sense of the present disclosure, aqueous-alcoholic solutions are to be understood to be aqueous solutions containing about 3 to about 70% by weight of a C₁-C₄ alcohol, especially ethanol or isopropanol. The inventive compositions may additionally contain further organic solvents such as methoxybutanol, benzyl alcohol, ethyl diglycol or 1,2-propylene glycol. All water-soluble organic solvents are preferred in this connection.

Methyl, ethyl, propyl, isopropyl, n-butyl, s-butyl and t-butyl, n-pentyl, i-pentyl, hexyl and n-hexyl are examples of such a C₁-C₆ alkyl group. Examples of a C₂-C₆ alkenyl group are vinyl, allyl, but-2-enyl, but-3-enyl and isobutenyl, vinyl and allyl being preferred C₂-C₆ alkenyl radicals.

In the course of the work leading to various embodiments contemplated herein, it has been found that it is advantageous if dyes contain at least one compound of the Formula (I) in which Y represents —NH₂.

It is particularly preferred if the R1 radical represents a group of the formula (II) wherein n is 4, 5 or 6. Also preferred are compositions, which contain at least one compound of the Formula (I), in which R1 is a group of Formula (III), m=1 or 2 and p=2 or 3. Compounds of Formula (I), in which R1 represents one of the groups —(CH₂)—O—(CH₂)₂—, —(CH₂)—O—(CH₂)₃—, —(CH₂)₂—O—(CH₂)₂—, —(CH₂)₂—O—(CH₂)₃—, are particularly preferred.

Compositions, which contain at least one compound of Formula (I), in which the R1 group represents a group of Formula (IV) with m=1 or 2 and p=2 or 3, are also particularly preferred.

Particularly preferred are compounds of formula (I), in which R1 represents a —(CH₂)—N(R3)-(CH₂)₂—, —(CH₂)—N(R3)-(CH₂)₃—, —(CH₂)₂—N(R3)-(CH₂)₂—, —(CH₂)₂—N(R3)-(CH₂)₃— group.

In the last named formulas, R3 preferably represents H or methyl, so that particularly preferred materials contain at least one compound of Formula (I), in which the R1 group represents one of the groups —(CH₂)—NH—(CH₂)₂—, —(CH₂)—NH—(CH₂)₃—, —(CH₂)₂—NH—(CH₂)₂, —(CH₂)₂—NH—(CH₂)₃, —(CH₂)—N(CH₃)—(CH₂)₂—, —(CH₂)—N(CH₃)—(CH₂)₃—, —(CH₂)₂—N(CH₃)—(CH₂)₂—, —(CH₂)₂—N(CH₃)—(CH₂)₃—.

Especially preferred compounds of Formula (I) are symmetrical, so that preferred inventive materials are characterized in that they contain at least one compound of Formula (I), in which the R1 and R2 groups are identical.

The compounds of Formula (I) are amino compounds. From these, the well-known acid addition salts can be produced in the usual way. All statements in this document and, accordingly, the claimed protection range relate both to the compounds in free form and also to their physiologically tolerated salts of organic or inorganic acids. Examples of such salts are the hydrochlorides, the hydrobromides, the sulfates, the phosphates, the acetates, the propionates, the citrates and the lactates. The hydrochlorides and the sulfates are particularly preferred.

The monohydrochlorides, the dihydrochlorides and the trihydrochlorides are particularly preferred in accordance with the present disclosure.

A preferred embodiment of the first object of the present disclosure is characterized in that the composition contains at least one compound of Formula (I), which is selected from
a) 4,6-dipyrrolidine-1-ylpyrimidine-2,5-diamine and/or
b) 4,6-bis(1-piperidyl)pyrimidine-2,5-diamine and/or
c) 4,6-bis(azepan-1-yl)pyrimidine-2,5-diamine and/or
d) 4,6-di(oxazolidine-3-yl)pyrimidine-2,5-diamine and/or
e) 4,6-dimorpholinopyrimidine-2,5-diamine and/or
f) 4,6-bis(1,3-oxazinan-3-yl)pyrimidine-2,5-diamine and/or
g) 4,6-bis(1,4-oxazepan-4-yl)pyrimidine-2,5-diamine and/or
h) 4,6-di(imidazoline-1-yl)pyrimidine-2,5-diamine and/or
i) 4,6-di(piperazine-1-yl)pyrimidine-2,5-diamine and/or
j) 4,6-di(hexahydropyrimidine-1-yl)pyrimidine-2,5-diamine and/or
k) 4,6-bis(1,4-diazepan-1yl)pyrimidine-2,5-diamine and/or
l) 4,6-bis(3-methylimidazolidine-1-yl)pyrimidine-2,5-diamine and/or
m) 4,6-bis(4-methylpiperazine-1-yl)pyrimidine-2,5-diamine and/or
n) 4,6-bis(3-methylhexahydropyrimidine-1-yl)pyrimidine-2,5-diamine and/or
o) 4,6-bis(4-methyl-1,4-diazepan-1-yl)pyrimidine-2,5-diamine and/or
p) mixtures and/or physiologically tolerated salts of these compounds.

Materials, preferred in accordance with the present disclosure, are characterized in that they contain compounds of Formula (I) and/or its physiologically tolerated salts in a ratio by weight of about 0.001 to about 5.0% by weight, preferably from about 0.025 to about 2.5% by weight, especially preferred from about 0.05 to about 2.0% by weight and particularly preferred from about 0.1 to about 1.5% by weight, based on the total weight of the ready-to-use material.

Materials, preferred in accordance with the present disclosure, contain additionally to the compound(s) of Formula (I), at least one coupler component. Particularly beautiful dyeing can be achieved if, as coupling component, at least one coupling component is contained in the materials, which are selected from the group of 3-amino-2-methylamino-6-methoxypyridine, 3-amino-6-methylphenol, 3-amino-2-hydroxypyridine, 1,3-bis-(2,4-diaminophenoxy)propane, 2,7-dihydroxynaphthaline, 2-methylresorcinol, 2,5-dimethylresorcinol, 4-chlororesorcinol, 3-aminophenol, 2-amino-3-hydroxypyridine, 2-chlor-6-methyl-3-aminophenol, 2,6-dizhydroxy-3,4-dimethylpyridine, 2-({3-[(2-hydroxyethyhamino]-4-methoxy-5-methylphenyl}amino)ethanol, 2-({3-[(2-hydroxyethyhamino]-2-methoxy-5-methylphenyl}amino)ethanol, 1-methoxy-2-amino-4-beta-hydroxy-ethylaminobenzene (Lehmanns blue), 2,4-diaminophenoxyethanol, 5-amino-4-chloro-o-cresol, 2,4-dichloro-m-aminophenol, 2,6-dihydroxy-3,4-dimethylpyridine and/or a physiologically tolerated salt of these compounds.

Likewise materials, preferred as contemplated herein, are characterized in that they contain the at least one coupling component and/or its physiologically tolerated salt in an amount of about 0.001 to about 5.0% by weight, more preferably from about 0.025 to about 2.5% by weight, particularly preferred from about 0.05 to about 2% by weight and in particular from about 0.1 to about 1.5% by weight, based in each case on the total weight of the ready-to-use material.

In order to achieve a balanced and subtly nuanced formation, it is advantageous as contemplated herein if further color-imparting components are contained in the inventive material. In accordance with the present disclosure, it may therefore be preferred if the material contains at least one further color-imparting component, which is selected from additional oxidation dye precursors of the developer type and/or substantive dyes.

Aside from the oxidation dye precursors of the developer type of Formula (I), the inventive material may contain at least one further developer component.

Specially preferred additional developer components are selected from at least one compound of the group comprising p-phenylendiamine, p-toluylenediamine, 2-(2-hydroxyethyl)-p-phenylendiamine, 2-(1,2-dihydroxyethyl)-p-phenylendiamine, N,N-bis-(2-hydroxyethyl)-p-phenylen-diamine, 2-methoxy-methyl-p-phenylendiamine, N-(4-amino-3-methylphenyI)—N-[3-(1H-imidazole-1-yl)propyl]amine, N,N′-bis-(2-hydroxyethyl)-N,N′-bis-(4-aminophenyl)-1,3-diamino-2-propanol, bis-(2-hydroxy-5-amino-phenyl)methane, 1,3-bis-(2,5-diaminophenoxy)-2-propanol, N,N′-bis-(4-amino-phenyl)-1,4-diazacycloheptane, 1,10-bis-(2,5-diaminophenyl)-1,4,7,10-tetraoxadecane, 4-amino-phenol, 4-amino-3-methylphenol, 4-amino-2-aminomethylphenol, 4-amino-2-(1,2-dihydroxyethyl)-phenol and 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidinw, 2-hydroxy-4,5,6-triaminopyrimidine, as well as the physiologically tolerated salts of these compounds. Especially preferred additional developer components are p-toluylenediamine, 2-(2-hydroxyethyl)-p-phenylenediamine, 2-methoxymethyl-p-phenylendiamine, N-(4-amino-3-methylphenyI)—N-[3-(1H-imidazole-1-yl)propylamine, and/or 4,5-diamino-1-(2-hydroxyethyl)-pyrazole as well as their physiologically tolerated salts.

The additional developer components preferably are used in an amount of about 0.0001 to about 10% by weight and especially in an amount of about 0.001 to about 5% by weight, based on the ready-to-use material.

Furthermore, the inventive material may contain at least one substantive dye. These are dyes, which dye the hair directly and do not require an oxidative process for forming the color. Substantive dyes usually are nitrophenylenediamines, nitroaminophenols, azo dyes, anthraquinones or indophenols.

Substantive dyes preferably are used in an amount of about 0.001 to about 20% by weight and especially of about 0.05 to about 5% by weight, based on the whole of the preparation used. The total amount of substantive dyes preferably does not exceed about 3% by weight.

Substantive dyes may be divided into anionic, cationic and nonionic substantive dyes, which are selected and used by someone of ordinary skill in the art in accordance with the requirements of the carrier base.

Preferred anionic substantive dyes are the compounds known under the international or commercial names of bromophenol blue, tetrabromophenol blue, acid yellow 1, yellow 10, acid yellow 23, acid yellow 36, acid orange 7, acid red 33, acid red 52, pigment red 57:1, acid blue 7, acid green 50, acid violet 43, acid black 1 und acid black 52.

Preferred cationic substantive dyes are basic blue 7, basic blue 26, basic violet 2 und basic violet 14, basic yellow 57, basic red 76, basic blue 16, basic blue 347 (cationic blue 347/dystar), basic blue 99, basic brown 16 and basic brown 17 as well as yellow 87, basic orange 31 and basic red 51.

Nonionic nitro and quinone dyes and neutral azo dies are particularly suitable as nonionic substantive dyes. Preferred nonionic substantive dyes are those compounds known under the international or commercial names of HC yellow 2, HC yellow 4, HC yellow 5, HC yellow 6, HC yellow 12, HC orange 1, disperse orange 3, HC red 1, HC red 3, HC red 10, HC red 11, HC red 13, HC red BN, HC blue 2, HC blue 11, HC blue 12, disperse blue 3, HC violet 1, disperse violet 1, disperse violet 4, disperse black 9, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol, 2-(2-hydroxyethyl)amino-4,6-dinitro-phenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)-amino-4-nitrobenzene, 2-[(4-amino-2-nitrophenyl)amino]-benzoic acid, 6-nitro-1,2,3,4-tetra-hydroquinoxaline, 2-hydroxy-1,4-naphtho-quinone, picramic acid and the salts thereof, 2-amino-6-chloro-4-nitrophenol, 4-ethylamino-3-nitro-benzoic acid and 2-chloro-6-ethylamino-4-nitrophenol.

Aside from the compound of Formula (I), the inventive material may also contain dyes analogous to natural dyes. The use of inventive compositions, which contain precursors of dyes, analogous to natural dyes as air oxidizable dyes is preferred. In this embodiment, said compositions are consequently not mixed with an additional oxidizing agent.

The precursors of dyes, analogous to natural dyes, preferably are used in an amount of about 0.001 to about 5% by weight based on the total application preparation. Particularly suitable as precursors of hair dyes, analogous to natural dyes, are derivatives of 5,6-dihydroxyindolines, especially 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline as well as 5,6-dihydroxyindoline-2-carboxylic acid and other derivatives of 5,6-dihydroxyindole, especially 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindol-2-carboxylic acid, as well as physiologically tolerated salts of the compounds named above.

In the case of oxidative dyeings, the color may be developed basically with oxygen from the air. Preferably however, a chemical oxidizing agent is used, especially if, aside from the dyeing, a brightening effect of the human hair is desired. This brightening effect may be desired independently of the dyeing method. As oxidizing agents, persulfates, peroxydisulfates, chlorites, hypochlorites and especially hydrogen peroxide and/or a solid addition product of an organic or inorganic compound come into consideration.

In order to prevent premature, undesirable reaction of the precursor of the oxidation dye with the oxidizing agent, precursors of the oxidation dye precursors and the oxidizing agent itself advisably are packaged separately and only brought into contact immediately before use.

Therefore, in a further embodiment of the present disclosure, materials are preferred, which are characterized in that they are prepared immediately before their application by mixing at least two preparations, the at least two preparations being provided in at least two separately packaged containers, wherein one container contains a dye (A), which contains at least one compound a) and at least one compound b) in a cosmetic carrier, and a further container contains an oxidizing agent preparation (B) containing at least one oxidizing agent.

Preferably, the oxidizing agent preparation (B) contains hydrogen peroxide and/or one of its solid addition products to organic or inorganic compounds, such as urea, melamine and sodium borate as oxidizing agent.

Preferably, the amount of oxidizing agent in the ready-to-use material is about 0.5 to about 12% by weight, especially about 2 to about 10% by weight and, particularly preferred, about 3 to about 6% by weight (calculated as 100% hydrogen peroxide) in each case related to the ready-to-use material.

In a further preferred embodiment, the inventive material is a material for dyeing and optionally, at the same time, for brightening keratinic fibers and preferably contains about 0.5 to about 15% by weight, especially about 1 to about 12.5% by weight, particularly about 1.5 to about 10% by weight and more particularly about 2 to about 6% by weight of hydrogen peroxide, in each case based on the total weight of the ready-to-use material.

Such preparations of oxidizing agent preferably are aqueous and flowable. Preferred preparations are characterized in that the flowable preparation of oxidizing agent contains about 40 to about 90% by weight, preferably about 50 to about 85% by weight, especially about 55 to about 80% by weight, particularly about 60 to about 77.5% by weight and more particularly about 65 to about 75% by weight of water.

However, in accordance various embodiments with the present disclosure, the oxidation dye may also be applied on the hair together with a catalyst, which activates the oxidation of the dye precursor. Such catalysts are, for example, certain enzymes, iodides, quinones or metal ions.

Furthermore, it has proven to be advantageous if the oxidizing agent preparations contain at least one stabilizer or complexing agent. Conventionally and preferred within the scope of the present disclosure, complexing agents and stabilizers are, for example, polyoxycarboxylic acids, polyamines, ethylenediaminetetraacetic acid (EDTA), N-hydroxyethylethylendiamine triacetic acid, diethylenetriamine pentaacettic acid (DTPA), ethylendiamine disuccinic acid (EDDS), hydroxyethyliminodiacetic acid, nitridodiacetic acid-3-propionic acid, isoserine diacetic acid, N,N-Di-(2-hydroxyethyl)glycine, N-(1,2-dicarboxy-2-hydroxyethyl)glycine, N-(1,2-dicarboxy-2-hydroxy-ethyl aspartic acid or nitrilotriacetic acid (NTA), ethylendiamindiglutaric acid (EDGA), 2-hydroxypropylendiamine disuccinic acid (HPDS), glycinamide-N,N′-disuccinic acid (GADS), ethylendiamine-N—N′-diglutaric acid (EDDG), 2-hydroxypropylenediamine-N—N′-disuccinic acid (HPDDS), diaminoalkyldi-(sulfosuccinic acid) (DDS), ethylenedicysteic acid (EDC), ethylenediamine-N—N′-bis(ortho-hydroxyphenyl)acetic acid (EDDHA), N-2-hydroxyethylamine-N,N-diacetic acid, glyceryliminodiacetic acid, iminodi(acetic acid)-N-2-hydroxypropylsulfonic acid, (aspartic acid)-N-carboxymethyl-N-2,5-hydroxypropyl-3-sulfonic acid, β-alanine-N,N′-diacetic acid, (aspartic acid)-N,N′-diacetic acid, (aspartic acid)-N-monoacetic acid, dipicolinic acid, as well as their salts and/or derivatives, geminal diphosphonic acids such as 1-hydroxyethane-1,1-diphosphonic acid (HEDP), the homologues thereof with up to 8 carbon atoms as well as hydroxy- or amino group-containing derivatives hereof and 1-aminoethane-1,1-diphosphonic acid, the higher homologues thereof with up to 8 carbon atoms as well as hydroxy- or amino group-containing derivatives, aminophosphonic acid such as ethylenediaminetetra(methylenephosphonic acid (EDTMP), diethylene-triaminepenta(methylenephosphonic acid) (DTPMP) as well as the higher homologues thereof or nitrilo-tri(methylenephosphonic acid), phosphonopolycarboxylic acids such as 2-phosphonobutane-1,2,4-tricarboxylic acid, cyclodextrins, as well as alkali stannates (sodium stannate), alkali pyrophosphates (tetrasodium pyrophosphate, disodium pyrophosphate), alkali phosphate (sodium phosphate) and phosphoric acid and its salts.

At alkaline pH values of the treating solutions, these complexing agents are present at least partly as anions. It does not matter whether they are introduced in the form of acids or in the form of salts. If they are used as salts, alkali, ammonium or alkyl ammonium salts, especially sodium salts are preferred.

Complexing agents, preferred in accordance with the present disclosure, are nitrogen-containing polycarboxylic acids, especially EDTA and phosphonates, preferably hydroxyalkane or amino-alkane phosphonates and, in particular 1-hydroxyethane-1,1-diphosphonate (HEDP) or the disodium or tetrasodium salt thereof and/or ethylenediaminetetramethylene phosphonate (EDTMP) or the hexasodium salt thereof, and/or diethylenetriaminepentamethylene phosphonate (DTPMP) or the heptasodium or octasodium salt thereof.

The dyeing preparation and, optionally, the oxidizing agent preparation contain further auxiliary materials and additives. In accordance various embodiments with the present disclosure, it has proven to be preferable if the dyeing preparation and/or the oxidizing agent preparation contain at least one thickening agent. There are no basic limitations with respect to these thickening agents. Organic as well as strictly inorganic thickening agents may be used.

In accordance with a first, preferred embodiment, the thickening agent is an anionic, synthetic polymer. Carboxylate and sulfonate groups are preferred anionic groups.

Examples of anionic monomers, of which the polymeric, anionic thickening agents consist, are acrylic acid, methacrylic acid, crotonic acid, itaconic acid, maleic anhydride and 2-acrylamido-2-methylpropanesulfonic acid. The acidity groups may be present partly or completely as the sodium, potassium, ammonium, monoethanolamine or triethanolamine salt. Maleic anhydride, as well as, in particular, 2-acrylamido-2-methylpropane-sulfonic acid and acrylic acid are preferred monomers.

Polyacrylic acids, which may or may not be cross-linked, are preferred anionic homopolymers. In this connection, allyl ethers of pentaerythritol, sucrose and propylene are preferred cross-linking agents. Such Compounds are obtainable commercially, for example, under the trade name of Carbopol®. Likewise preferred is the homopolymer of 2-acrylamido-2-methylpropane-sulfonic acid, which is obtainable commercially, for example, under the name of Rheothik® 11-80.

Within this first embodiment, it may be preferred to use copolymers of at least one anionic monomer and at least one non-ionic monomer. With respect to the anionic monomers, references made to the substances listed above. Preferred non-ionic monomers are acrylamide, methacrylamide, acrylate ester, methacrylate ester, the monoesters and diesters of itaconic acid, vinylpyrrolidone, vinyl ether and vinyl esters.

The anionic acrylic acid and/or methacrylic acid polymers or copolymers are contained in the inventive materials preferably in an amount of about 0.1 to about 10% by weight and especially of about 1 to about 5% by weight, based in each case on the weight of the material.

Preferred anionic copolymers are, for example, copolymers of acrylic acid, methacrylic acid or the C₁-C₆ alkyl esters thereof, as sold under the INCI declaration of acrylate copolymers. A preferred commercial product is, for example, Aculyn® 33 of Rohm & Haas. However, copolymers of acrylic acid, methacrylic acid or the C₁-C₆ alkyl esters and the esters of an ethylenically unsaturated acid and an alkoxylated fatty alcohol are furthermore preferred. Suitable ethylenically unsaturated acids are, in particular, acrylic acid, methacrylic acid and itaconic acid; suitable alkoxylated fatty alcohols are, in particular, steareth-20 or ceteth-20. Such copolymers are sold by Rohm & Haas under the trade name of Aculyn® 22 and by National Starch under the trade names of Structure® 2001 and Structure® 3001.

Preferred anionic copolymers furthermore are acrylic acid-acrylamide copolymers and, in particular, polyacrylamide copolymers with monomers, which contain sulfonic acid groups. A particularly preferred anionic copolymer consists of about 70 to about 55 mole % of acrylamide and about 30 to about 45 mole % of 2-acrylamido-2-methylpropanesulfonic acid, wherein the sulfonic acid group is present partly or completely as the sodium, potassium, ammonium, monoethanolammonium or triethanolammonium salt. This polymer may also be present in cross-linked form, wherein preferably polyolefinically unsaturated compounds, such as tetraallyoxythane, allyl sucrose, allyl pentaerythritol and methylene bis acrylamide are used. One such polymer is contained in the commercial products, Sepigel® 305 and Simulgel® 600 of SEPPIC. The use of these compounds, which contain a mixture of hydrocarbons (C₁₃-C₁₄ isoparaffin or isohexadecane) and a non-ionic emulsifier (laureth-7 or polysorbate-80) aside from the polymer components, has proven to be particularly advantageous within the scope of the inventive teachings.

Polymers of maleic anhydride and methyl vinyl ether, especially those with cross-linking, are preferred thickening agents. A maleic acid/methyl vinyl ether copolymer, cross-linked with 1,9-decadiene, is commercially available under the name of Stabileze® QM.

Preferably, the inventive material may contain at least one anionic acrylic acid and/or methacrylic acid polymer or copolymer. Preferred polymers of this type are:

Polymers, for example, of at least about 10% by weight of a lower alkyl ester of acrylic acid, about 25 to about 70% by weight of methacrylic acid and, optionally, up to about 40% by weight of a further comonomer,

Mixed polymers of about 50 to about 75% by weight of ethyl acrylate, about 25 to about 35% by weight of acrylic acid and about 0 to about 25% by weight of other comonomers are known. Suitable dispersions of this type are commercially available, for example, under the trade name of Latekoll® D (BASF).

Copolymers of about 50% to about 60% by weight of ethyl acrylate, about 30 to about 40% by weight of methacrylic acid and about 5 to about 15% by weight of acrylic acid, cross-linked with ethylene glycol dimethacrylate.

In accordance with a first, embodiment, the thickening agent is a cationic, synthetic polymer. Quaternary ammonium groups are preferred cationic groups. Especially those polymers, for which the quaternary ammonium group is linked over a C₁-C₄ hydrocarbon group to a polymer chain, which is built up from acrylic acid, methacrylic acid or the derivatives thereof, have proven to be very suitable. Homopolymers of the general Formula (HP-1),

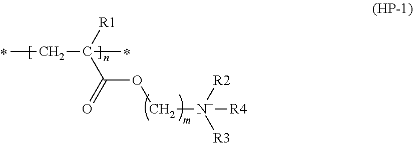

in which R1=—H or —CH₃, R2, R3 and R4 independently from one another, are selected from C₁-C₄-alkyl-, -alkenyl- or

hydroxyalkyl groups, m=1, 2, 3 or 4, n is a natural number and X⁻ is a physiologically tolerated, organic or inorganic anion, as well as copolymers consisting essentially of the monomer units listed in Formula (HP-1), as well as non-ionic monomer units, are particularly preferred cationic, polymeric gel-forming agents. Within the scope of these polymers, those are preferred as contemplated herein, for which at least one of the following conditions applies:

R1 represents a methyl group

R2, R3 and R4 represent methyl groups

m has a value of 2.

As physiologically tolerated counter ions X—, halide irons, sulfate ions, phosphate ions, methosulfate ions, as well as organic ions such as lactate, citrate, and acetate ions come into consideration. Halide ions, especially chloride ions, are preferred.

A poly(methacryloxy-ethyltrimethylammonium chloride), cross-linked if desired and having the INCI name of polyquarternium-37, is a particularly suitable homopolymer. The cross linking may be carried out, if desired, with the help of olefinic polyunsaturated compounds, such as divinyl benzene, tetraallyloxyethane, methylene bisacrylamide, diallylether, polyallyl-polyglyceryl ether or allyl ethers of sugars or sugar derivatives such as erythritol, pentaerythritol, arabitol, mannitol, sorbitol, sucrose or glucose. Methylene bisacrylamide is a preferred cross-linking agent.

The homopolymer preferably is used in the form of a nonaqueous polymer dispersion, the polymer portion of which should not be less than about 30% by weight. Such polymer dispersions are commercially obtainable under the names of Salcare® SC 95 (about 50% polymer, further components: mineral oil (INCI name: mineral oil) and tridecyl-polyoxypropylene-polyoxyethylene ether (INCI name: PPG-1 trideth-6)) and Salcare® SC 96 (with a polymer portion of about 50%, further components: mixture of diesters of propylene glycol with a mixture of caprylic acid and capric acid (INCI name: propylene glycol dicaprylate/dicaprate) and tridecyl-polyoxy-propylene-polyoxyethylene ether (INCI name: PPG-1-trideceth-6). Copolymers with monomer units of formula (HP-1) contain monomer units, preferably acrylamide, methacrylamide, C₁-C₄ alkyl esters of acrylic acid and methacrylic acid as nonionic monomer units. Of these non-ionic monomers, acrylamide is preferred. As in the case of the homopolymers described above, these copolymers also may be cross-linked. The cross-linked acrylamide methacroyl-oxyethyltrimethylammonium chloride copolymer is a copolymer preferred in accordance with the present disclosure. Such copolymers, in which the monomers may be present in a ratio by weight of about 20:80, are commercially obtainable as an about 50% nonaqueous polymer dispersion under the name of Salcare® SC 92.

In a further preferred embodiment, naturally occurring thickening agents are used. Preferred thickeners of this embodiment are, for example, non-ionic guar gums. In accordance with various embodiments the present disclosure, modified as well as unmodified guar gums may be used. Unmodified guar gums are sold, for example, under the trade name of Jaguar® by Rhone Poulenc. Modified guar gums, preferred in accordance with the present disclosure, contain C₁-C₆ hydroxyalkyl groups. The hydroxymethyl, hydroxyethyl, hydroxypropyl and hydroxybutyl groups are preferred. Guar gums, so modified, are known in the prior art and may be prepared, for example, by the reaction of guar gums with alkylene oxides. The degree of hydroxyalkylation, which corresponds to the number of alkylene oxide molecules in proportion to the number of free hydroxyl groups of the guar gum, preferably is between 0.4 and 1.2. Such modified guar gums are commercially available under the trade name of Jaguar® HP8, Jaguar® HP60, Jaguar® HP120, Jaguar® DC 293 and Jaguar® HP105 from Rhone Poulenc.

Further, suitable natural thickeners are also already known from the prior art.

Furthermore, biosaccharide gums of microbial origin, such as the scleroglucan gums or xanthan gums, gums from plant exudates, such as gum arabic, gum ghatti, karaya gum, gum tragacanth, carrageenan, agar, carob bean gum, pectins, alginates, starch fractions and derivatives such as amylose, amylopectin and dextrins, cellulose derivatives such as methylcellulose, carboxyalkylcelluloses and hydroxy alkyl celluloses, are preferred in accordance with this embodiment.

Especially the hydroxyethylcelluloses, which are sold under the names of Cellosize® by Amerchol and Natrosol® by Hercules, are preferred hydroxyalkylcelluloses. Especially the carboxymethylcelluloses, as sold under the names of Blanose® by Aqualon, Aquasorb® and Ambergum® by Hercules and Cellgon® by Montello, are suitable carboxyalkyl celluloses.

Starch and its derivatives are furthermore preferred. Starch is a storage material of plants, which occurs, particularly, in bulbs and roots, in grain seeds and in fruit and can be obtained in high yield from a large number of plants. The polysaccharide, which is insoluble in cold water and forms a colloidal solution in boiling water, may be obtained, for example, from potatoes, cassava, sweet potatoes, corn, grains, rice, legumes such as peas, beans, bananas or the pulp of certain varieties of palm (such as sago palms). Natural starches, obtained from plants and/or chemically or physically modified starches, may be used in accordance with the present disclosure. A modification may be obtained, for example, by introducing different functional groups at one or more of the hydroxyl groups of the starch. Usually, modified starches are esters, ethers or amides of the starch with optionally substituted C₁-C₄₀ groups. A corn starch, etherified with a 2-hydroxypropyl group, as sold, for example, by National Starch under the commercial name of Amaze®, is particularly advantageous. However, non-ionic, fully synthetic polymers, such as polyvinyl alcohol or polyvinylpyrrolidinones, may also be used as inventive thickeners. Preferred, non-ionic, fully synthetic polymers are sold, for example, by BASF under the trade name of Luviskol®. Aside from their outstanding thickening properties, such as nonionic polymers also enable the sensory feel of the resulting preparations to be improved clearly.

As inorganic thickeners, phyllosilicates (polymeric, crystalline sodium di silicates) have proven to be particularly suitable in the sense of the present disclosure. Especially clays, particularly magnesium aluminum silicates such as bentonite, particularly smectites such as montmorillonite or hectorite, which optionally may also be modified suitably, and synthetic phyllosilicates, such as the magnesium phyllosilicate, sold under the name of Optigel® by Sad Chemie, are preferred.

In order to increase the performance of the preparation of oxidizing agent even further, at least one silica compound, which may be hydrated, may be added to the inventive composition. In accordance with the present disclosure, it may be preferred to use the optionally hydrated silica compounds in the amounts of about 0.05% by weight to about 15% by weight, amounts of about 0.15% by weight to about 10% by weight being particularly preferred and amounts of about 0.2% by weight to about 5% by weight being more particularly preferred, the amounts in each case being based on the inventive, anhydrous composition. The amounts given reflect the content of the silica compounds (without their water content) in the materials.

The present disclosure is basically not subject to any limitations with regard to the optionally hydrated silica compounds. Silicic acids, the oligomers and polymers thereof, as well as the salts thereof are preferred. The alkali salts, especially the potassium and sodium salts, are preferred salts. The sodium salts are particularly preferred.

The optionally hydrated silica compounds may be present in various forms. In accordance with the present disclosure, the silica compounds in the form of silica gels are preferred and those in the form of water glass are particularly preferred. The silica compounds may be present partly as an aqueous solution.

In accordance with the present disclosure, water glass is particularly preferred and is formed from a silicate of formula (SiO₂)ₙ(Na₂O)ₘ(K₂O)ₚ, wherein n represents a positive, rational number and m and p, independently of one another, represent a positive, rational number or 0, with the proviso that at least one of the parameters m or p is not 0 and the ratio between n and the sum of m and p is between 1:4 and 4:1. Meta-silicates, in which the ratio between n and the sum of m and p is 1.2 or less, are preferred.

Aside from the components described by the molecular formula, the water glass may contain slight amounts of other additives, such as phosphates or magnesium salts.

Water glass, which is particularly preferred as contemplated herein, is sold, for instance, by Henkel under the name of Ferrosil® 119, sodium and water glass 40/42, Portil® A, Portil® AW and Portil® W and by Akzo under the name of Britesil® C20.

Preferably, the preparation (A) and/or optionally the preparation of oxidizing agent (B) are fabricated as flowable preparations.

Preferably, an emulsifier or a surface active substance is furthermore added to the flowable preparations (A) and/or (B), surface active substances being described as surfactants or emulsifiers depending on the area of application and being selected from anionic, cationic, zwitterionic, amphoteric and nonionic surfactants and emulsifiers. These materials are described in detail in the following.

All anionic, surface active materials, suitable for use on the human body, are suitable as anionic surfactants in the inventive preparations. These are characterized by an anionic group, which makes them water soluble, such as, for example, a carboxylate, sulfate, sulfonate or phosphate group and a lipophilic alkyl group with 8 to 30 carbon atoms. In addition, glycol ether or polyglycol ether groups, ester, ether and amide groups as well as hydroxyl groups may be contained in the molecule.

Alkyl sulfates, alkyl ether sulfates and ether carboxylic acids with 10 to 18 carbon atoms in the alkyl group and up to 12 glycol ether group in the molecule are preferred anionic surfactants.

Those surface active compounds are referred to as zwitterionic surfactants, which contain at least one quaternary ammonium group and at least one carboxylate, sulfonate or sulfate group in the molecule. The so-called betaines, such as the N-alkyl-N,N-dimethylammonium glycinate, for example the cocoalkyl-dimethyl-ammonium glycinate, N-acyl-aminopropyl-N,N-dimethylammonium glycinates, for example, the cocoacylaminopropyl-dimethylammonium glycinate, and 2-alkyl-3-carboxymethyl-3-hydroxyethyl imidazoline with 8 to 18 carbon atoms in the alkyl or acyl group as well as the cocoacyl-aminoethylhydroxyethylcarboxymethyl glycinate are particularly suitable zwitterionic surfactants. The fatty acid amide derivative, known under the INCI name of cocoamidopropyl betaine, is a preferred zwitterionic surfactant.

Those surface active compounds, which, aside from a C₈-C₂₄ alkyl or acyl group in the molecule, contain at least one free amino group and at least one —COOH or SO₃H group and are capable of forming an internal salt, are understood to be amphoteric surfactants. N-alkylglycine, N-alkylpropionic acids, N-alkylaminobutyric acid, N-alkyliminodipropionic acids, N-hydroxyethyl-N-alkylamidopropylglycine, N-alkyltaurine, N-alkylsarcosine, 2-alkylaminopropionic acid and alkylaminoacetic acids with in each case about 8 to 24 carbon atoms in the alkyl group, are examples of suitable amphoteric surfactants. The N-cocoalkylaminopropionate, the cocoacylaminoethylamino propionate and the C₁₂-C₁₈ acylsarcosine are particularly preferred amphoteric surfactants.

Furthermore, it has proven to be advantageous if the inventive dyes and brightening agents contain further nonionic, surface active materials. As hydrophilic group, nonionic surfactants contain, for example, a polyol group, a polyalkylene glycol ether group or a combination of a polyol group and a polyglycol ether group.

The anionic, non-ionic, zwitterionic or amphoteric surfactants are used in an amount of about 0.1 to about 45% by weight, preferably of about 1 to about 30% by weight and particularly of about 1 to about 15% by weight, based on the total amount of the ready-to-use material.

Cationic surfactants of the quaternary ammonium compound type, esterquats and amidoamines are also preferred as contemplated herein. Ammonium halides, especially chlorides and bromides, such as alkyl trimethylammonium chlorides, dialkyl dimethyl ammonium chlorides and trialkyl methyl ammonium chlorides, such as cetyltrimethylammonium chloride and stearyltri-methylammonium chloride, di stearyldimethylammonium chloride, lauryldimethylammonium chloride, lauryldimethylbenzylammonium chloride and tricetylmethylammonium, as well as imidazolium compounds, known under the INCI names of quaternium-27 and quaternium-83, are preferred quaternary ammonium compounds. The long chain alkyl groups of the surfactants named above preferably have 10 to 18 carbon atoms. The quaternized protein hydrolysates are further cationic surfactants, which can be used as contemplated herein.

The cationic surfactants are present in the inventive compositions in amounts of about 0.05 to about 10% by weight, based on the total composition. Amounts of about 0.1 to about 5% by weight are particularly preferred.

In a preferred embodiment, nonionic, zwitterionic and/or amphoteric surfactants and their mixtures may be preferred.

In a further preferred embodiment, the effect of the inventive active ingredient may be increased by emulsifiers. Such emulsifiers are, for example:

addition products of 4 to 30 moles of ethylene oxide and/or 0 to 5 moles of propylene oxide to linear fatty alcohols having 8 to 22 carbon atoms, to fatty acids having 12 to 22 carbon atoms and to alkylphenols containing 8 to 15 carbon atoms in the alkyl group,

C₁₂-C₂₂-fatty acid mono- and diesters of addition products of 1 to 30 moles of ethylene oxide to polyols having 3 to 6 carbon atoms, in particular to glycerol,

Ethylene oxide and polyglycerol addition products to methylglucoside fatty acid esters, fatty acid alkanolamides and fatty acid glucamides,

C₈-C₂₂ alkylmono- and oligoglycosides and their ethoxylated analogues, with degrees of oligomerization of about 1.1 to about 5, in particular of about 1.2 to about 2.0, and glucose as the sugar component being preferred,

mixtures of alkyl (oligo)glucosides and fatty alcohols, for example, the commercially available product Montanov® 68,

addition products of 5 to 60 moles of ethylene oxide onto castor oil and hydrogenated castor oil,

partial esters of polyols having 3 to 6 carbon atoms with saturated fatty acids having 8 to 22 carbon atoms,

sterols, which are understood to mean a group of steroids, which carry a hydroxyl group at carbon atom 3 of the steroid skeleton and are isolated from animal tissue (zoosterols) as well as from vegetable fats (phytosterols). Examples of zoosterols are cholesterol and lanosterol. Examples of suitable phytosterols are ergosterol, stigmasterol and sitosterol. Sterols, the so-called mycosteriols, are also isolated from fungi and yeasts.

Phospholipids, especially glucose phospholipids, which are used, for example, as lecithins or phosphatidyl cholines, for example, from egg yolk or plant seeds (such as soybeans),

fatty acid esters of sugars and sugar alcohols, such as sorbitol

polyglycerols and polyglycerol derivatives, for example, polyglycerol poly-12-hydroxystearate (commercial product Dehymuls® PGPH)

linear and branched fatty acids with 8 to 30 carbon atoms and the sodium, potassium, ammonium, calcium, magnesium and zinc salts thereof

The inventive materials contain the emulsifiers preferably in amounts of about 0.1 to about 25% by weight and particularly of about 0.5 to about 15% by weight, based on the total weight of the ready-to-use material.

Nonionic emulsifiers or surfactants having an HLB value of about 10-about 15 may be particularly preferred as contemplated herein. Among the emulsifier types mentioned, the emulsifiers which contain no ethylene oxide and/or no propylene oxide in the molecule may be very particularly preferred.

Furthermore, the inventive compositions may contain further active ingredients, auxiliaries and additives, such as

nonionic polymers such as, for example, vinylpyrrolidinone/vinyl acrylate copolymers, polyvinylpyrrolidinone, vinylpyrrolidinone/vinyl acetate copolymers, polyethylene glycols and polysiloxanes;

Silicones such as volatile or non-volatile, linear, branched or cyclic, crosslinked or not crosslinked polyalkylsiloxanes (such as dimethicones or cyclomethicones), polyarylsiloxanes and/or polyalkylarylsiloxanes, in particular polysiloxanes having organofunctional groups, such as substituted or unsubstituted amines (amodimethicones), carboxyl, alkoxy and/or hydroxyl groups (dimethicone copolyols), linear polysiloxane (A) polyoxyalkylene (B) block copolymers, grafted silicone polymers with a non-silicone-containing organic basic frame or with a polysiloxane basic frame, such as the commercial product Abil B 8832, sold under the INCI name Bis-PEG/PPG-20/20 dimethicone by Degussa, or mixtures thereof; cationic polymers such as quaternized cellulose ethers, polysiloxanes having quaternary groups, dimethyldiallylammonium chloride polymers, acrylamide-dimethyldiallyl ammonium chloride copolymers, diethyl sulfate quaternized dimethylaminoethyl methacrylate-vinylpyrrolidinone copolymers, vinylpyrrolidinone-Imidazolinium-methochloride copolymere and quaternized polyvinyl alcohol;

zwitterionic and amphoteric polymers such as, for example, acrylamidopropyl-trimethylammonium chloride/acrylate copolymers and octylacrylamide/methyl methacrylate/t-butylaminoethyl methacrylate/2-hydroxypropyl methacrylate copolymers, diallyldimethyl-ammonium chloride/acrylate-copolymere, t-butylaminoethylmethacrylate/N-(1,1,3,3-tetramethyl-butyl)acrylamide/acrylate(/methacrylate) copolymers,

anionic polymers such as, for example, polyacrylic acids, crosslinked polyacrylic acids, vinyl acetate/crotonic acid copolymers, vinylpyrrolidinone/vinyl acrylate copolymers, vinyl-acetate/butyl maleate/isobornyl acrylate copolymers, methylvinylether/maleic anhydride copolymers and acrylic acid/ethyl acrylate/N-t-butyl acrylamide terpolymers,

other thickeners such as agar-agar, guar gum, alginates, xanthan gum, gum arabic, karaya gum, locust bean gum, linseed gum, dextranes, cellulose derivatives, such as methylcellulose, hydroxyalkylcellulose and carboxymethylcellulose, starch fractions and derivatives such as amylose, amylopectin and dextrins, clays such as, for example, bentonite or fully synthetic hydrocolloids such as, for example, polyvinyl alcohol,

structurants such as glucose, maleic acid and lactic acid

hair-conditioning compounds such as phospholipids, for example, for example soy lecithin, egg lecithin and cephalins, as well as silicone oils,

paraffin oils, dimethyl isosorbide and cyclodextrins,

solvents and solubilizers, such as ethanol, isopropanol, ethylene glycol, propylene glycol, glycerin and diethylene glycol,

fiber structure-improving substances, in particular mono-, di- and oligosaccharides such as, for example, glucose, galactose, fructose, fruit sugar and lactose,

quaternized amines, such as methyl-1-alkylamidoethyl-2-alkylimidazolinium methosulfate,

defoamers such as silicones,

dyes for coloring the material,

active anti-danddruff ingredients such as piroctones, olamines, zinc omadines and climbazole,

amino acids and oligopeptides, in particular arginine and/or serine,

protein hydrolysates on an animal and/or vegetable basis, such as elastin, collagen, keratin, silk and milk protein hydrolysates, or almond, rice, pea, potato and wheat protein hydrolysates, and in the form of their fatty acid condensation-products or, where appropriate, anionically or cationically modified derivatives,

vegetable oil, such as macadamia nut oil, coconut oil, palm oil, amaranthseed oil, peach kernel, avocado oil, olive oil, coconut oil, rapeseed oil, sesame oil, jojoba oil, soybean oil, peanut oil, evening primrose oil and tea tree oil,

light stabilizers, in particular derivatized benzophenones, cinnamic acid derivatives and triazines,

substances for adjusting the pH, such as, for example, convential acids, especially food acids and bases,

active ingredients such as panthenol, pantothenic acid, pantolactone, allantoin, pyrrolidinonecarboxylic acids and salts thereof, and bisabolol,

polyphenols, in particular hydroxycinnamic acids, 6,7-dihydroxycoumarins, hydroxybenzoic acids, catechins, tannins, leukoanthocyanidins, anthocyanidines, flavanones, flavones and flavonols;

ceramides, preferably the sphingolipids, such as ceramides I, ceramides II, ceramides 1, ceramides 2, ceramides 3, ceramides 5 and ceramides 6, or pseudoceramides, such as, in particular, in particular, N—(C₈-C₂₂-acyl)-cyclohexyl) hydroxyproline,

Vitamins, provitamins and vitamin precursors, in particular those of groups A, B₃, B₅, B₆, C, E, F and H,

plant extracts such as the extracts of aloe vera, angelica, anise, apricot, benzoin, bergamot, birch, nettle, calmus, cassis, costus, marshmallow, oak bark, elemi, tarragon, spruce needle, galbanum, geranium, ginseng, grapefruit, guajak wood, green tea, hamamelis, haunchle, hops, coltsfoot, ginger root, iris, jasmine, chamomile, cardamon, clover, burr root, pine, kiwi, coconut, coriander, cumin, lavender, lemongrass, lily, lime, linden bleed, litchi, macis, mallow, almond, mango, melissa, melon, meristem, myrrh, neroli, olibanum, opoponax, orange, patchouli, petitgrain, pine, thyme, rooibos, roses, rosemary, horse-chestnut, sandalwood, sage, horsetail, yarrow, celery, fir, thyme, juniper, vine leaves, hawthorn, wheat, cuckoo flower, ylang-ylang, cedar and lemon,

Cholesterol,

consistency enhancers such as sugar esters, polyol esters or polyol alkyl ethers,

fats and waxes such as spermaceti, beeswax, montan wax and paraffins,

fatty acid alkanolamides,

swelling agents and penetrants such as glycerin, propylene glycol monoethylether, carbonate, hydrogen carbonate, guanidine, urea, as well as primary, secondary and tertiary phosphates,

opacifiers such as latex, styrene/PVP copolymers and styrene/acrylamide copolymers

pearlescing agents such as ethylene glycol mono- and distearate and PEG-3 distearate,

Pigments,

stabilizing agents for hydrogen peroxide and other oxidizing agents.

propellants such as propane-butane mixtures, N₂O, dimethyl ether, carbon dioxide and air,

antioxidants.

These additional substances will be selected by someone of ordinary skill in the art in accordance with the desired properties of the compositions.

With regard to further optional components as well as the amounts of these components used, reference is made expressly to the relevant handbooks, for example Kh. Schrader, Grundlagen and Rezepturen der Kosmetika (Fundamentals and Formulations of Cosmetics), 2^{nd} edition, Hüthig Buch Verlag, Heidelberg, 1989, which are know to someone of ordinary skill in the art.

The additional active ingredients and additives preferably are used in the inventive compositions in amounts of about 0.0001 to about 10% by weight and, in particular, of about 0.0005 to about 5% by weight, based on the total weight of the application mixture.

For the strong brightening of very dark hair, the use of hydrogen peroxide alone or of its addition products to organic or inorganic compounds is frequently not sufficient. The inventive compositions therefore also contain additional blonding and/or bleaching agents.

If, in addition to the dyeing of the keratinic fiber, a strong brightening is desired, a blonding preparation (C), containing at least one bleaching activator, preferably is mixed in addition with the mixture of oxidizing agent preparation (B) and the preparation (A) containing at least one oxidation dye precursor of formula (I).

It may be immaterial whether a mixture of (A) and (B) is prepared first and the bleaching composition (C) is then admixed or a different sequence of mixing the individual components is used. Preferably, the individual preparations are mixed as closely as possible to the chronological sequence and the ready-to-use agent preferably is applied to the keratinic fibers in a timely manner.

A further embodiment of the present application therefore is a material for bleaching and coloring keratinic fibers, characterized in that it is prepared by mixing at least one oxidizing agent preparation (B), containing at least one oxidizing agent, selected from hydrogen peroxide, the addition compounds thereof on solid carriers, at least one blonding preparation (C), containing at least one bleaching enhancer and at least one preparation (A), containing at least one oxidation dye precursor of Formula (I) in a cosmetic carrier.

In a further embodiment, it is preferred if the inventive dying agent additionally contains at least one inorganic peroxy compound as a bleaching preparation (C). Preferably, the inorganic peroxy compound is selected from ammonium persulfate, alkali metal persulfates, ammonium peroxymonosulfate, alkali metal hydrogen peroxymonosulfates, alkali metal peroxydiphosphates and alkaline earth metal peroxides. Organic peroxy compounds, particularly preferred as bleaching strength enhancers are ammonium peroxydisulfate, potassiumperoxydisulfate, sodium peroxydisulfate, potassium hydrogen peroxymono-sulfate, potassium peroxydiphosphate, magnesium peroxide and barium peroxide, in particular ammonium peroxydisulfate, potassium peroxydisulfate and sodium peroxydisulfate.

The inorganic peroxy compounds preferably are present in an amount of from about 0.1 to about 25% by weight and, in particular, in an amount of from about 0.5 to about 15% by weight, based on the total weight of the ready-to-apply material.

The persulfate salts or peroxydisulfate salts generally are used in the form of an optionally dust-free powder or of a compressed molded body.

However, it may be advantageous in accordance with various embodiments of the present disclosure if the material is free of inorganic peroxy compounds. Instead of or in addition to the solid peroxy compounds, the inventive material may contain a further bleaching reinforcer.

In the context of this present disclosure, as additional bleaching reinforcing agents, compounds, which, under perhydrolysis conditions, give aliphatic peroxycarboxylic acids, for example polyacylated alkylenediamines, in particular tetraacetylethylenediamine (TAED) and/or substituted perbenzoic acid, carbonic acid derivatives; for example, ammonium hydrogen carbonate, ammonium carbonate, sodium hydrogen carbonate, sodium carbonate, potassium hydrogen carbonate, potassium carbonate, calcium carbonate, alkyl carbonates and carbamates, as well as silyl carbonates and carbamates may be used.

The bleaching reinforcers, used alongside or instead of peroxy compounds, preferably are present in inventive cosmetic compositions in amounts of about 0.05 to about 10% by weight and, in particular, in amounts of about 0.2 to about 5% by weight, based in each case on the total weight of the ready-to-use material.

Although, in principle, there are no restrictions with regard to the formulation of the blonding preparation (C), it has proven to be preferable in accordance with various embodiments of the present disclosure to formulate preparation (C) so that it is anhydrous.

In the context of the present disclosure, anhydrous means a water content, based on the preparation (C), of less than about 5% by weight and, in particular, of less than about 2% by weight.

Blonding preparations, which contain less than about 0.1% by weight of water, may be particularly preferred in accordance with an exemplary embodiment of the present disclosure. Preferably, preparation (C) is formulated as a powder or as an anhydrous paste.

In a further preferred embodiment, the material in preparation (C) may contain at least one cationic pyridinium derivative as a bleaching reinforcer. Inventive materials, which contains at least one compound from 2-acetyl-1-methylpyridinium p-toluenesulfonate and/or 4-acetyl-1-methyl-pyridinium p-toluenesulfonate and /methyl-3,4-dihydroisoquinolinium p-toluenesulfonate as a cationic pyridinium derivative, are particularly preferred.

An embodiment of the present disclosure, preferred in accordance with the present disclosure, consists therein that the ready-to-use material has a pH of between about 7 and about 11, especially of between about 8 and about 10.5 and particularly between about 8.5 and about 10.0.

Usually, the pH is adjusted with pH adjusting means. Someone of ordinary skill in the art of cosmetics is familiar with current acidification and alkalizing materials. The alkalizing agents, which are suitable for adjusting the pH, typically are selected from inorganic salts, in particular from the alkali and alkaline earth metals, organic alkalizing agents, in particular, amines, basic amino acids and alkanolamines, and ammonia. Consumable acids, such as, for example, citric acid, acetic acid, malic acid or tartaric acid and diluted mineral acids are preferred acidifying agents in accordance with various embodiments of the present disclosure.

The pH values of the present disclosure were measured at a temperature of about 22° C.

Organic alkalizing agents, which can be used as contemplated herein, preferably are selected from alkanolamines of primary, secondary or tertiary amines with a C₂-C₆ basic alkyl group, which carries at least one hydroxyl group. Especially preferred alkanolamines are selected from the group comprising 2-amino-1-ethanol (monoethanolamine), 2-amino-2-methyl-1-propanol, 2-amino-2-methyl-1,3-propanediol and triethanolamine.

However, it has been found within the scope of the investigations of the present disclosure, that furthermore preferred agents are characterized in that they additionally contain an inorganic alkalizing agent. The inventive, inorganic alkalizing agent preferably is selected from the group comprising sodium hydroxide, potassium hydroxide, calcium hydroxide, barium hydroxide, sodium phosphate, potassium phosphate, sodium silicate, potassium silicate, sodium carbonate and potassium carbonate. Sodium hydroxide and/or potassium hydroxide are particularly preferred.

The basic amino acids, which can be used as the alkalinizing agents of the present disclosure, are preferably selected from the group comprising L-arginine, D-arginine, D/L-arginine, L-lysine, D-lysine and D/L-lysine, L-arginine, D-arginine, D/L-arginine being particularly preferred as alkalizing agent in the context of various embodiments.

Finally, ammonium hydroxide is a further preferred alkalizing agent.

The alkalizing agents preferably are present in an amount of about 0.05 to about 10% by weight and, in particular, of about 0.5 to about 5% by weight, based in each case on the total weight of the ready-to-use material.

As already mentioned, the inventive compositions may also be prepared directly, before they are applied from two or more separately packaged preparations. This is particularly suitable for separating incompatible components in order to avoid a premature reaction. A separation of multicomponent systems is particularly suitable where incompatibilities of the constituents are to be expected or feared. In such systems, the ready-to-use material is prepared by the consumer by mixing the components directly prior to use. A dye and/or brightening agent, in which the oxidation dye precursors initially are present separately from the oxidizing agent preparation and which preferably contain hydrogen peroxide, is preferred.

A preferred form of administering the inventive composition is a packaging unit (kit-of-parts), which, in containers that are made separately from one another

contains at least one preparation (A) in a container A containing, at least one oxidation dye precursor of Formula (I) in a cosmetic carrier and

at least one oxidizing agent preparation (B) in a container B containing at least one oxidizing agent in a cosmetic carrier.

If a particularly strong brightening effect is desired, a preferred further form of administering the inventive composition is a packaging unit (kit-of-parts), which is produced with separate containers

a container A containing at least one preparation (A), which contains at least one compound a) and at least one compound b) in a cosmetic carrier,

a container B containing at least one oxidizing agent preparation (B), which contains at least one oxidizing agent and

a container C containing at least one blonding preparation (C), which contains at least one bleaching reinforcer.

Preferably, the multicomponent packing unit (kit-of-parts) additionally contains instructions for use. Furthermore, it may be preferred if an application aid, such as a comb or a brush, and/or personal protective equipment, such as, for example, disposable gloves, is included with the kit.

With respect to further preferred embodiments of the multi-component packaging unit (kit-of-parts), what has been stated in regard to the inventive material applies mutatis mutandis.

The actual hair dye advisably is prepared immediately before use by mixing the preparations (A) with (B) and optionally (C). The application temperatures may range from about 15 to about 40° C. After a period of action of about 5 to about 45 minutes, the hair dye is removed by rinsing it from the hair that is to be dyed. Washing afterwards with a shampoo is omitted, if a dyeing shampoo with a high concentration of surfactant in the carrier, was used.

During the period of action of the material on the fibers, it may be advantageous if the dyeing process is supported by heat. The heat supplied may be controlled by an external heat source, such as the warm air of a blower, as well as, particularly in the case when the hair of a living test person is dyed, by the body temperature of said test person. For the latter possibility, the part to be colored is usually covered with a hood. In particular, the temperature during the time of action is between about 10° C. and about 45° C. and especially between about 20° C. and about 40° C. The inventive dyeing agents produce intensive dyeings already at physiologically tolerated temperatures of below about 45° C. They are therefore particularly suitable for dyeing human hair.

A further subject matter of the present disclosure is the use of an inventive composition in dyeing agents for human hair to improve the gray coverage, the leveling, the color intensity, the durability and/or the color fullness of the dyeing results.

With respect to further preferred embodiments of the inventive use, what has been stated with regard to the inventive material applies mutatis mutandis.

### Examples

### 1) Synthesis Examples

### i) 4,6-Bis(piperidine-1-yl)pyrimidine-2,5-diamine, dihvdrochloride (E1)

### (1) Synthesis of 2-Amino-4,6-dimethoxypyrimidine

To a solution of 15.0 g (0.652 mol) of sodium in 400 ml of methanol, 32.8 g (0.200 mol) of 2-amino-4,6-dichloropyrimidine were added, and the mixture was refluxed for 13 h with stirring and stirred at room temperature for 3 days. At the end of the reaction, the precipitated sodium chloride was removed and the solution was concentrated to dryness. The residue was washed with 500 ml of water. After drying, 2-amino-4,6-dimethoxypyrimidine (26.5 g, 85%) was obtained as a pale yellow solid.

M.P.: 96-101° C.

¹H-NMR (300 MHz, d₆-DMSO): δ=3.80 (s, 3H, 6-0Me), 5.38 (s, 1H, 5-H), 6.59 (s, 2H, NH2).

¹³C-NMR (125 MHz, d₆-DMSO): δ=53.2 (6-0Me), 77.9 (5-C), 162.8 (2-C), 171.7 (6-C, 4-C).

### (2) Synthesis of 2-Amino-4,6-dimethoxy-5-nitrosopyrimidine

To a solution of 41.9 g (0.270 mol) of 2-amino-4,6-dimethoxypyrimidine in 675 ml of dimethyl sulfoxide, 34.8 g (0.297 mol) of isopentyl nitrite were added and stirred at room temperature for 96 hours. At the end of the reaction, the deep blue solution was poured into 2.51 of water and stirred at room temperature for 1 hour and the resulting precipitate was filtered off with suction. The residue was washed with water. After drying, 2-amino-4,6-dimethoxy-5-pyrimidine (32.8 g, 66%) was obtained as a light blue solid.

M.P.: 215-218° C. (dec.)

¹H-NMR (300 MHz, d₆-DMSO): δ=3.94 (s, 3H, 6-0Me), 8.28 (s, 2H, NH₂).

¹³C-NMR (125 MHz, d₆-DMSO): δ=54.9 (6-0Me), 141.9 (5-C), 163.3 (6-C, 4-C), 173-0 (2-C).

### (3) Synthesis of 5-Nitroso-4,6-bis(1-piperidyl)pyrimidine-2-amine

To a solution of 1.84 g (10.0 mmol) of 2-amino-4,6-dimethoxy-5-nitrosopyrimidine in 50 ml of ethanol, 1.70 g (20.0 mol) of piperidine were added and stirred at room temperature for 78 hours. At the end of the reaction, the violet solution was concentrated. After drying, 5-nitroso-4,6-bis(1-piperidyl)pyrimidine-2-amine (2.50 g, 86%) was obtained as a violet solid.

¹H-NMR (300 MHz, d₆-DMSO): δ=1.59 (m, 6H, 2′-CH2 and 3′-CH2), 3.92 (m, 4H, CH2), 7.11 (s, 2H, NH2).

¹³C-NMR (125 MHz, d₆-DMSO): δ=24.2 (3′-C), 26.7 (2′-C), 52.1 (1′-C), 140.8 (5-C), 151.3 (2-C), 162.5 (4-C), 165.3 (6-C).

### (4) Synthesis of 4,6-Bis(piperidine-1-yl)pyrimidine-2,5-diamin (E1)

To a solution of 2.03 g (7.00 mmol) of 5-nitroso-4,6-bis (piperidin-1-yl)pyrimidin-2-amine in ethanol (400 ml), 0.5 g (5%) of palladium on charcoal was added and shaken in a hydrogen atmosphere at atmospheric pressure for 4 hours at room temperature. At the end of the reaction, the reaction solution was poured onto 12.2 ml of hydrochloric acid (35 mmol, 10% in water), the catalyst was filtered off and the solution was concentrated almost to dryness. The precipitate was filtered off with suction and then washed with little ice cold ethanol. After drying, 4,6-bis (piperidin-1-yl)pyrimidine-2,5-diamine dihydrochloride (2.48 g, 96%) was obtained as a brown solid.

¹H-NMR (300 MHz, d₆-DMSO): δ=1.53 (m, 6H, 2′-CHz and 3′-CHz), 2.93 (m, 4H, l′-CHz), 9.14 (s, 2H, NH₂).

¹³C-NMR (125 MHz, d₆-DMSO): δ=22.1 (3′-C), 22.4 (2′-C), 43.8 (1′-C), 140.8 (5-C), 151.3 (2-C), 162.5 (4-C), 165.3 (6-C).

### ii) 5-Methyl-4,6-dimorpholino-pyrimidine-2-amine. dihydrochloride (E2)

### (1) Synthesis of 4,6-Dimorpholino-5-nitroso-pyrimidin-2-amine

To a solution of 1.84 g (10.0 mmol) of 2-amino-4,6-dimethoxy-5-nitrosopyrimidine in 50 ml of ethanol, 1.74 g (20.0 mol) of morpholine in 22 ml of acetonitrile were added and stirred at room temperature for 5 days. At the end of the reaction, the violet solution was concentrated to half its volume. The precipitate obtained was filtered off and re-crystallized from ethanol (220 ml). After drying, 4,6-dimorpholino-5-nitrosopyrimidine-2-amine (1.25 g, 42%) was obtained as a violet solid.

¹H-NMR (300 MHz, d₅-DMSO): δ=2.82 (d, 4H, I—CH2), 3.70 (d, 4H, 2′-CH2), 8.48 (s, 2H, NH2).

¹³C-NMR (125 MHz, d₅-DMSO): δ=44.8 (1′-C), 51.7 (2′-C), 52.1 (1′-C), 140.7 (5-C), 151.5 (2-C), 162.4 (4-C), 165.9 (6-C).

### (2) Synthesis of 5-Methyl-4,6-dimorpholino-pyrimidine-2-amine dihydrochloride (E2)

To a solution of 1.62 g (5.50 mmol) of 4,6-dimorpholino-5-nitroso-pyrimidine-2-amine in ethanol (400 ml), 0.20 g (5%) of palladium on charcoal was added and shaken in a hydrogen atmosphere at atmospheric pressure and room temperature for 24 hours. At the end of the reaction, the reaction solution was poured onto 9.6 ml of hydrochloric acid (27.5 mmol, 10% in water), the catalyst was filtered off and the solution was concentrated almost to dryness. The precipitate was filtered off with suction and then washed with little ice cold ethanol. After drying, 5-methyl-4,6-dimorpholino-pyrimidine-2-amine dihydrochloride (1.63 g, 82%) was obtained as a brown solid.

¹H-NMR (300 MHz, d₅-DMSO): δ=3.03 (d, 2H, 1′-CH3), 3.89 (d, 2H, 2′-CH2), 8.73 (brs, 4H, NH2).

¹³C-NMR (125 MHz, d₅-DMSO): δ=42.9 (1′-C), 63.4 (2′-C), 84.8 (5-C), 152.0 (4-C, 6-C), 157.4 (2-C).

### 2) Dyeings

### Preparation of the Color Cream

A collar cream of the following composition was prepared.

| | | |
|---|---|---|
| Hydrenol ® D¹ | 8.5% | by weight |
| Lorol ® tech.² | 2.0% | by weight |
| Texapon ® NSO³ | 20.0% | by weight |
| Dehyton ® K⁴ | 12.5% | by weight |
| Eumulgin ® B2⁵ | 0.75% | by weight |
| Sodium sulfite | 1.0% | by weight |
| Ammonium sulfate | 1.0% | by weight |
| Developer component | 3 | mmol |
| Coupler component | 3 | mmol |

| | |
|---|---|
| Water | ad 100 |

¹C₁₆-₁₈ fatty alcohol (INCI name: Cetearyl alcohol) (Cognis)
²C₁₂-₁₈ fatty alcohol (INCI name: Coconut alcohol) (Cognis)
³Lauryl ether sulfate, sodium salt (ca. 27.5% active substance; INCI name: Sodium Laureth Sulfate) (Cognis)
⁴N,N-Dimethyl-N-(C₈₋₁₈-cocoamidopropyhammonium)acetobetaine (ca. 30% active substance; INCI name: Aqua (Water), Cocamidopropyl Betaine) (Cognis)
⁵Cetylstearylalkohol with ca. 20 EO units (INCI name: Ceteareth-20) (Cognis)

Hydrenol D and Lorol techn. were melted together with Texapon NSO, Dehyton K and Eumulgin B2 at 80° C. The melt was then emulsified with the sodium sulfite and ammonium sulfate dissolved in a portion of the water. The inventive developer was dissolved in a further part of the stated amount of water with heating and added with stirring. The coupling agent was also dissolved in a portion of the stated amount of water and added with stirring. The formulation was then filled up to 100% with water and stirred cold.

The color cream, obtained in this manner, was mixed in a 1:1 ratio with the following developer dispersion, which contained 6% hydrogen peroxide.

| | | |
|---|---|---|
| Dipicolinic acid | 0.1 | percent by weight |
| Sodium pyrophosphate | 0.03 | percent by weight |
| Turpinal ® SL⁶ | 1.50 | percent by weight |
| Texapon ® N28⁷ | 2.00 | percent by weight |
| Acrysol ® 22⁸ | 0.60 | percent by weight |
| Hydrogen peroxide, 50% | 6.00 | percent by weight |
| sodium hydroxide, 45% | 0.80 | percent by weight |
| Water | ad 100 | percent by weight |

⁶1-Hydroxyethane-1,1-diphosphonic acid (approx. 58-61% active substance content; INCI-Name: Etidronic Acid, Aqua (Water)) (Solutia)
⁷sodium salt of lauryl ether sulfate (at least 26.5% active substance content; name: Sodium Laureth Sulfate) (Cognis)
⁸Acrylpolymer (approx. 29.5-30.5% solids and water; INCI name: Acrylate/Steareth-20 Methacrylate Copolymer)

For the dyeing process, the fourfold amount of the ready-to-use mixture was applied on a strand of 80% greyed hair (Kerling). After a period of action of 30 minutes at 32° C., the strands were rinsed and washed with a conventional shampoo. The dyeing of the strands was evaluated visually after drying under a daylight lamp. The results of the dyeing are summarized in the following Table.

E1: Dyeings with 4,6-Bis(piperidine-1-yl)pyrimidine-
2,5-diamine, Dihydrochloride

| | | Shade | Color |
|---|---|---|---|
| Example | Coupling component | obtained | intensity |
| 2 | 3-Amino-2-methylamino-6- | olive | +++ |
| | methoxypyridine | | |
| 3 | 5-Amino-2-methylphenol | ivory | + |
| 4 | 3-Aminophenol | brownorange | ++ |
| 5 | 2-Methylresorcin | greyred | ++ |

+++ High intensity
++ Average intensity
+ Low intensity

E2: Dyeings with 5-Methyl-4,6-dimorpholino-
pyrimidine-2-amine Dihydrochloride

| | | Shade | Color |
|---|---|---|---|
| Example | Coupling component | obtained | intensity |
| 1 | Resorcinol | flesh colored | + |
| 2 | 3-Amino-2-methylamino-6- | olive | +++ |
| | methoxypyridine | | |
| 3 | 5-Amino-2-methylphenol | ivory | + |
| 4 | 1,3-Bis(2,4-diaminophe- | chocolate brown | +++ |
| | noxy)propane | | |
| 5 | 2-Methylresorcinol | brown orange | ++ |
| 6 | 3-Aminophenol | brown orange | ++ |

+++ High intensity
++ Average intensity
+ Low intensity

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A composition for dyeing keratinic fibers, the composition comprising, in a cosmetic carrier, at least one neutral tetraaminopyrimidine derivative of the general Formula (I): 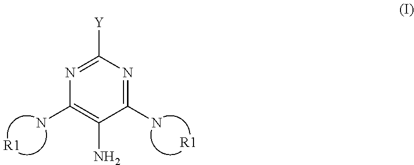
in which
R1, R2 independently of one another, represent one of the groups of formulas (II), (III) or (IV)
—(CH2)n- (II)
—(CH2)m-O—(CH2)p- (III)
—(CH2)m-N(R3)-(CH2)p (IV)
n represents 2, 3, 4, 5 or 6,
m, p independently of one another represent 1, 2 or 3,
Y represents —OH, —NH2 or —NH-alkyl,
R3 represents a hydrogen atom, a C1-C6 alkyl group or a C2-C6 alkenyl group.

2. The composition according to claim 1, wherein the composition comprises at least one compound of Formula (I), in which Y represents —NH2.

3. The composition according to claim 1, wherein the composition comprises at least one compound of Formula (I), in which the R1 and R2 groups are identical.

4. The composition according to claim 1, wherein the composition comprises at least one compound of Formula (I), in which the R1 represents a group of Formula (II) with n=4, 5 or 6.

5. The composition according to claim 1, wherein the compound(s) of Formula (I) is/are selected from
a) 4,6-dipyrrolidine-1-ylpyrimidine-2,5-diamine and/or
b) 4,6-bis(1-piperidyl)pyrimidine-2,5-diamine and/or
c) 4,6-bis(azepan-1-yl)pyrimidine-2,5-diamine and/or
d) 4,6-di(oxazolidine-3-yl)pyrimidine-2,5-diamine and/or
e) 4,6-dimorpholinopyrimidine-2,5-diamine and/or
f) 4,6-bis(4-methylpiperazine-1-yl)pyrimidine-2,5-diamine and/or
g) 4,6-bis(1,4-oxazapan-4-yl)pyrimidine-2,5-diamine and/or
h) 4,6-di(imidazolidine-1-yl)pyrimidine-2, 5-diamine and/or
i) 4,6-di(piperazine-1-yl)pyrimidine-2,5-diamine and/or
j) 4,6-di(hexahydropyrimidine-1-yl)pyrimidine-2,5-diamine and/or
k) 4,6-bis(1,4-diazepan-1-yl)pyrimidine-2,5-diamine and/or
l) 4,6-bis(3-methylimidazolidine-1-yl)pyrimidine-2, 5-diamine and/or
m) 4,6-bis(4-methylpiperazine-1-yl)pyrimidine-2,5-diamine and/or
n) 4,6-bis(3-methylhexahydropyrimidine-1-yl)pyrimidine-2,5-diamine and/or
o) 4,6-bis(4-methyl-1,4-diazepan-1-yl)pyrimidine-2,5-diamine and/or
p) mixtures and/or physiologically tolerated salts of these compounds.

6. The composition according to claim 1, wherein the composition comprises at least one coupler component selected from the group consisting of 3-amino-2-methylamino-6-methoxypyridine, 3-amino-6-methylphenol, 3-amino-2-hydroxypyridine, 1,3-bis(2,4-diaminophenoxy)propane, 2,7-dihydroxynaphthalene, 2-methylresorcinol, 2,5-dimethylresorcinol, 4-chlororesorcinol, 3-aminophenol, 2-amino-3-hydroxypyridine, 2-chloro-6-methyl-3-aminophenol, 2,6-dihydroxy-3,4-dimethylpyridine, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 1-methoxy-2-amino-4-beta-hydroxy-ethylaminobenzene (Lehmann's blue), 2,4-diaminophenoxyethanol, 5-amino-4-chloro-o-cresol, 2,4-dichloro-m-aminophenol, 2,6-dihydroxy-3,4-dimethylpyridine and/or a physiologically tolerated salt of these compounds.

7. The composition according to claim 1, wherein the composition comprises the compound(s) of Formula (I) in an amount of from about 0.001 to about 5.0% by weight, based in each case on the total weight of the ready-to-use composition.

8. The composition according to claim 6, wherein the at least one coupler component and/or the physiologically tolerated salt thereof is present in an amount of from about 0.001 to about 5.0% by weight, based in each case on the total weight of the ready-for-use composition.

9. The composition for dyeing and/or brightening keratinic fibers according to claim 1, wherein the composition comprises from about 0.5 to about 15% by weight of hydrogen peroxide, based on the total weight of the ready-to-use composition.

10. A method comprising using a composition according to claim 1 in dyes for human hair for improving gray coverage, leveling, color intensity, and/or durability of dyeings.

11. The composition according to claim 1, wherein the composition comprises the compound(s) of Formula (I) in an amount of from about 0.025 to about 2.5% by weight, based on the total weight of the ready-to-use composition.

12. The composition according to claim 1, wherein the composition comprises the compound(s) of Formula (I) in an amount of from about 0.05 to about 2.0% by weight, based on the total weight of the ready-to-use composition.

13. The composition according to claim 1, wherein the composition comprises the compound(s) of Formula (I) in an amount of from about 0.1 to about 1.5% by weight, based on the total weight of the ready-to-use composition.

14. The composition according to claim 6, wherein the at least one coupler component and/or the physiologically tolerated salt thereof is present in an amount of from about 0.025 to about 2.5% by weight, based on the total weight of the ready-for-use composition.

15. The composition according to claim 6, wherein the at least one coupler component and/or the physiologically tolerated salt thereof is present in an amount of from about 0.05 to about 2.0% by weight, based on the total weight of the ready-for-use composition.

16. The composition according to claim 6, wherein the at least one coupler component and/or the physiologically tolerated salt thereof is present in an amount of from about 0.1 to about 1.5% by weight, based on the total weight of the ready-for-use composition.

17. The composition for dyeing and/or brightening keratinic fibers according to claim 1, wherein the composition comprises from about 1 to about 12.5% by weight of hydrogen peroxide, based on the total weight of the ready-to-use composition.

18. The composition for dyeing and/or brightening keratinic fibers according to claim 1, wherein the composition comprises from about 1.5 to about 10% by weight of hydrogen peroxide, based on the total weight of the ready-to-use composition.

19. The composition for dyeing and/or brightening keratinic fibers according to claim 1, wherein the composition comprises from about 2 to about 6% by weight of hydrogen peroxide, based on the total weight of the ready-to-use composition.
